(19) United States
(12) Patent Application Publication
(10) Pub. No.: US 2005/0101678 A1
(43) Pub. Date: May 12, 2005

(54) Antibacterial composition

(76) Inventor: Andreas Natsch, Uetikon (CH)

Correspondence Address: NORRIS, MCLAUGHLIN & MARCUS, PA, 875 THIRD STREET, 18TH FLOOR, NEW YORK, NY, 10022, US

(21) Appl. No.: 10/657,345

(22) Filed: Sep. 8, 2003

(30) Foreign Application Priority Data
Aug. 14, 2000 (EP) 00 117 496.0

(51) Int. Cl. A61K 31/075; A61K 31/045

(52) U.S. Cl. 514/715; 514/729

(57) ABSTRACT

The present invention relates to an antibacterial composition containing a compound of formula I 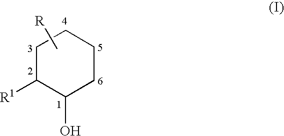
wherein R is a residue of the formula II 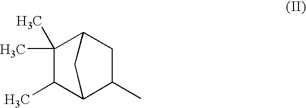
and R is located at position 2, 3, or 6, and R¹ is hydrogen; or R is located at position 4, and R¹ is hydrogen or methoxy; or R is located at position 5, and R₁ is methoxy. The present invention also relates to the use of the composition in personal care products and methods of making personal care products employing the composition. The antibacterial composition is active against Corynebacteria, Staphylococci, and Brevibacteria. It can therefore inhibit formation of different kinds of body malodor.

### FIELD OF THE INVENTION

The present invention relates to an antibacterial composition, its use in personal care products and methods of making personal care products containing the composition.

### BACKGROUND OF THE INVENTION

Body malodor is formed when fresh perspiration, which is odorless per se, is decomposed by bacteria such as Staphylococci and Corynebacteria, both genera belonging to the gram-positive Eubacteriaceae.

Cosmetic deodorants are intended to inhibit formation of axillary body malodor. They are based on different active principles. One principle is the suppression of perspiration by astringents. According to a second principle the bacterial flora on the skin is reduced by antibacterial substances. But many commercially used antibacterial compounds affect the entire microbial flora on the skin. In addition some agents have higher activity against the low-odor forming Staphylococci bacteria than against the Corynebacteria and may thus favor the latter, which is highly undesirable.

Foot deodorants and deodorants for shoes are used to prevent the formation of a typical cheesy smell of feet. This odor is formed by Brevibacteria under prolonged periods of humidity which may occur especially in individuals with excessive perspiration or in shoes with insufficient aeration.

Another skin manifestation attributed to a bacterial origin is acne, which is commonly treated with topically applied cosmetic products containing antibacterial agents.

Antibacterial compounds currently used in deodorant or antiperspirant products include for example Triclosan (2,4,4′-trichloro-2′-hydroxy-diphenyl-ether). However, the use of such chlorinated compounds as antibacterial agents is strongly questioned by consumer organizations.

Further, Triclosan is much more active for the low-odor forming Staphylococci then for the Corynebacteria, and application of low amounts of this compound may thus favor populations of Corynebacteria.

Antibacterial properties of certain odoriferous substances, essential oils, or other fragrance ingredients are known to be used to manufacture deodorizing fragrance compositions. One natural fragrance compound with particularly high activity is 3,7,11-trimethyl-2,6,10-dodecatrien-1-ol (“Farnesol”). Its use in deodorizing fragrance compositions has been described in DE 27 28 921 B2 and DE 33 15 058 A1. However, to obtain an antibacterial effect, higher levels than the ones customarily used in perfumery must be employed.

Another natural fragrance compound known for its antibacterial activity is Sandalwood oil. In a recent study by Viollon et Chaumont (Parfums & cosmetiques, 1994, 116:67-70) the antibacterial activity against axilla bacteria of 26 essential oils were compared. Among them Sandalwood oil has the strongest activity. However, high price and limited availability of this natural oil hamper its widespread use in commercial cosmetic products at the levels needed for a growth inhibition of skin bacteria.

### SUMMARY OF THE INVENTION

An object of the present invention is to provide a composition having an antibacterial effect against Corynebacteria.

Another object of the present invention is to provide a composition having an antibacterial effect against Staphylococci.

A further object of the present invention is to provide a composition having an antibacterial effect against Brevibacteria.

Another object of the present invention is to provide a composition having an antibacterial effect against Propionibacteria.

One embodiment of the present invention is an antibacterial composition containing a compound of formula I 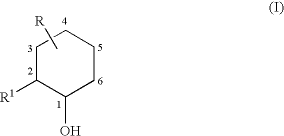
wherein R is a residue of formula II 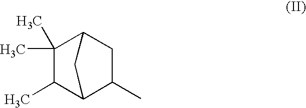
and R is located at position 2, 3, or 6, and R¹ is hydrogen; or R is located at position 4, and R¹ is hydrogen or methoxy; or R is located at position 5, and R¹ is methoxy.

Another embodiment of the present invention is a method of making a personal care product by admixing a personal care product with a perfume and a compound of formula I 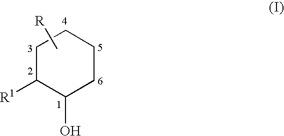
wherein R is a residue of formula II 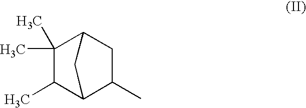
and R is located at position 2, 3, or 6, and R¹ is hydrogen; or R is located at position 4, and R¹ is hydrogen or methoxy; or R is located at position 5, and R¹ is methoxy.

A further embodiment of the present invention is a personal care product, a malodor inhibiting product, an acne inhibiting product, or a deodorant and/or antiperspirant product containing a compound of formula I 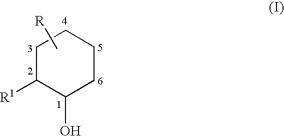
wherein R is a residue of formula II 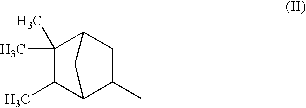
and R is located at position 2, 3, or 6, and R¹ is hydrogen; or R is located at position 4, and R¹ is hydrogen or methoxy; or R is located at position 5, and R¹ is methoxy.

### DETAILED DESCRIPTION OF THE INVENTION

The present invention is based on the surprising finding that among 250 commonly used fragrance compounds an antibacterial composition containing a compound of formula I 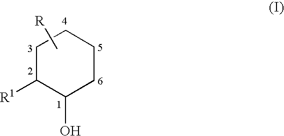
wherein R is a residue of the formula II 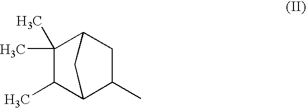
and R is located at position 2, 3, or 6, and R¹ is hydrogen; or R is located at position 4, and R¹ is hydrogen or methoxy; or R is located at position 5, and R¹ is methoxy, is very active against a collection of Corynebacterium sp. strains isolated from healthy human volunteers.

The composition exhibits an activity even slightly higher than that of Sandalwood oil, and only slightly below the activity of Farnesol. The activity of the compound is significantly higher than, e.g., the activity of Sandalore and Radjanol. It was shown that compounds according to the present invention offer both, outstanding olfactive properties as well as an excellent antibacterial activity. Furthermore the intensity of the antibacterial effect against Corynebacteria and Staphylococcus is equal, which is not the case for Triclosan.

The colorless viscous liquid Sandela is obtained by a two step reaction, wherein Camphene and Guaiacol are treated with an acidic catalyst followed by hydrogenation (Dorsky, et al., U.S. Pat. No. 3,499,937), and its preparation is relatively cheap. The above two step reaction results in a mixture of the compounds of formula I, i.e. 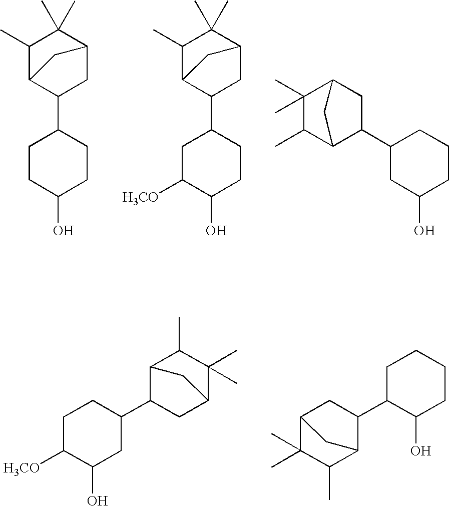
and all possible enantiomers. Each compound of formula I can be isolated by distillation or chromatography. The compounds may be used alone or in combination with each other in the composition of the present invention.

The highest activity against Corynebacteria was found for 2-methoxy-4-(5,5,6-trimethylbicyclo[2.2.1]hept-2-yl)cyclohexan-1-ol and 2-methoxy-5-(5,5,6-trimethyl-bicyclo[2.2.1]hept-2-yl)cyclohexan-1-ol. With 2-methoxy-4-(5,5,6-trimethylbicyclo[2.2.1]hept-2-yl)cyclohexan-1-ol an outstanding activity is achieved.

Another preferred embodiment of the composition according to the present invention is a composition, wherein the compound is 3-(5,5,6-trimethylbicyclo[2.2.1]hept-2-yl-)cyclohexan-1-ol.

The compound in the composition according to the present invention also has strong antibacterial properties against Brevibacterium epidermidis, the organism involved in formation of foot odor. The compound in the composition according to the present invention is an appropriate synthetic molecule to control this organism. Its activity is equal to that of natural Sandalwood oil and 2 to 6 times higher than the synthetics Radjanol and Sandalore. Therefore the compound in the composition according to the present invention can inhibit formation of-different kinds of body malodor.

Additionally, the compound in the composition according to the present invention has good antibacterial properties against Propionibacterium acnes as well. Therefore it is useful for prevention and treatment of acne.

Odoriferous substances are by definition volatile compounds that evaporate from the skin and therefore loose their deodorizing effect. Furthermore, small water-soluble fragrance compounds can get dissolved in sweat and are thus rapidly diluted under conditions of high sweat secretion, which further reduces the duration of the deodorizing effect. Therefore a low water solubility, as indicated by a high LogP value, and a low volatility, as indicated by low vapor pressure, are desired for odoriferous substances which are used for their antibacterial activity. Indeed it has been found that 2-(5,5,6-trimethylbicyclo[2.2.1]hept-2-yl)-cyclohexanol, 3-(5,5,6-trimethylbicyclo[2.2.1]hept-2-yl)-cyclohexanol, and 4-(5,5,6-trimethylbicyclo[2.2.1]hept-2-yl)-cyclohexanol, in addition to exhibiting pronounced antibacterial activity, meet these criteria. They have a vapor pressure of about 2.2 μg/L and a calculated LogP of about 5.27. In this respect they are similar to Farnesol, which has a vapor pressure of 1.35 μg/L and a calculated LogP value of 5.31. Compared to, e.g., the common antibacterial perfume ingredient Eugenol with a vapor pressure of 109 μg/L and a calculated LogP of 2.2 their deodorant activity is therefore maintained over a prolonged period of time when applied to the skin.

Since products that exert an antibacterial activity by means of individual fragrance compounds employ higher levels of these compounds than the ones customarily used in perfumery, these antibacterial compounds must meet several additional criteria. They should have a relatively low odor value, i.e. they should only be perceived when used at higher amounts to avoid materially affecting the olfactive balance of the perfume. The odor value is defined as the vapor pressure (in ng/L) divided by the olfactive threshold value (in ng/L) and it corresponds to the dilution factor above threshold (DFT). Commercial Sandela containing all isomers has an odor value of 265, whereas Sandalore has an odor value of 5685, Ebanol has an odor value of 212557, and the odor value of Radjanol is 53130. Santalol, the main active ingredient of Sandalwood oil, has an odor value of 650 and Eugenol has an odor value of 346904. The compounds of the present invention therefore meet the criterion of low odor value and their olfactive impact on the fragrance is much lower compared to other compounds with Sandalwood odor like Sandalore and Ebanol or other antibacterial perfume components like Eugenol. Thus they may be incorporated in quantities sufficiently high to obtain good antimicrobial activity in perfumes without affecting the overall impression too much. This would not be the case for other synthetic fragrance molecules with low thresholds like Ebanol and Sandalore. Finally, the sandalwood note of the compounds of the present invention is highly desirable as a base note for perfumes applied to the human body.

2-methoxy-4-(5,5,6-trimethylbicyclo[2.2.1]hept-2-yl)cyclohexan-1-ol and 2-methoxy-5-(5,5,6-trimethyl-bicyclo[2.2.1]hept-2-yl)cyclohexan-1-ol are odorless and can therefore be included in cosmetic formulations without affecting the odor.

The term “perfume” in the present invention means a mixture containing odoriferous substances and other ingredients that are known to those skilled in the art. In a preferred embodiment the composition according to the present invention contains a perfume, the latter comprising 10 to 80% by weight of the compound.

In another preferred embodiment the composition according to the present invention contains a perfume, the perfume having from about 10 to about 80% by weight of the compound as the only antibacterial agent.

The compositions according to the present invention require a compound level that is higher than the one used in customary perfumery. The recommended level in the final product should ideally be between 0.3% to 0.6% by weight. If the perfume level in the deodorizing or antiperspirant product is kept at 1% by weight, the perfume should contain between 30% and 60% by weight of the compound. If the perfume level in the product is 1.5% by weight, the perfume comprises preferably 20 to 40% by weight of the compound. However, to obtain stronger effects the use level may be increased to 1% by weight in the finished deodorant or antiperspirant product. Finally, if the compound is used in combination with other antibacterial fragrance compounds the level in the final product may be lowered to 0.1% by weight.

The compound in the composition according to the present invention does not need to be added to the perfume, but can also be added directly to the deodorant or antiperspirant product. In this case the compound is added separate from the perfume to the deodorant or antiperspirant formula at a level of 0.1 to 1% by weight.

In a preferred embodiment, the composition according to the present invention contains a perfume, about 10 to about 80% by weight of which perfume is composed of a compound of formula I.

In another preferred embodiment, the composition according to the present invention contains a perfume, the perfume having from about 10 to about 80% by weight of the compound as the only antibacterial agent.

The compound can be used for partially replacing Farnesol in antibacterial perfumes. Indeed, these two products can be combined in any ratio, and their antibacterial effect is additive. The invention thus further relates to the use of the compound to reduce the usage level of Farnesol. Due to the four times lower price of the compound compared to Farnesol, this results in significant cost savings and an improvement of the overall olfactive performance of the perfume. Like the compound, Farnesol can be added as an ingredient of the perfume or separately from the perfume. In antibacterial compositions containing a perfume, the perfume containing the compound and Farnesol, the level of the compound is preferably between about 10 and about 80% by weight, the level of Farnesol between about 5 and about 50% by weight.

The compound according to the present invention need not be employed only for cost savings and improved olfactive performance, it can be used to enhance the antibacterial effect of Farnesol as well. By maintaining normal levels of Farnesol, which is used as separate ingredient of the deodorant or antiperspirant product, and combining this with a perfume with high compound content, an enhanced antibacterial effect can be obtained. The invention thus also relates to the use of high levels of the compound as well as Farnesol in deodorant and antiperspirant products to obtain increased deodorant effects.

Compositions according to the present invention may contain, in addition to the compound of formula I, another ingredient. Such an ingredient may be water, dipropylene glycol, propylene glycol, or combinations thereof.

Compositions according to the present invention comprising the compound may be formulated in various forms such as deodorant stick, roll-on, pump-spray, aerosol, deodorant soap, powder, solution, gel, cream, stick, balm and lotion. As used herein, such products are called “personal care products”.

Another embodiment of the present invention is a method of making a personal care product. This method includes:
admixing a personal care product with a perfume and a compound of formula I 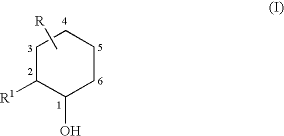
   wherein R is a residue of formula II 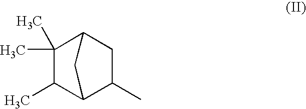
   and R is located at position 2, 3, or 6, and R¹ is hydrogen; or R is located at position 4, and R¹ is hydrogen or methoxy; or R is located at position 5, and R¹ is methoxy.

A further embodiment of the present invention is a personal care product, a malodor inhibiting product, an acne inhibiting product, or a deodorant and/or antiperspirant product containing a compound of formula I 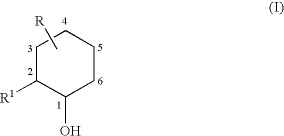
wherein R is a residue of formula II 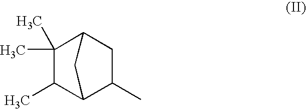
and R is located at position 2, 3, or 6, and R¹ is hydrogen; or R is located at position 4, and R¹ is hydrogen or methoxy; or R is located at position 5, and R¹ is methoxy.

The following examples are provided to further illustrate the compounds, compositions, and processes of the present invention. These examples are illustrative only and are not intended to limit the scope of the invention in any way.

### EXAMPLES

### Example 1

Comparison of the Antibacterial Activity of Compound of the Formula I with Other Perfume Ingredients and Antibacterial Agents when Tested Against Human Axilla Bacteria

The antibacterial efficacy of the relevant fragrance compounds in the present invention in microtiter plate tests is demonstrated below. The different bacterial strains had been isolated from the human axilla by current microbiological practice. They were taxonomically identified by cell morphology, gram-reaction, and biochemical tests included in the Api coryne test kit (BioMerieux, France). Strain Staphylococcus epidermidis Ax25 was identified by fatty acid methyl ester analysis (FAME; German type strain collection DSMZ, Germany). The strains were maintained on Tryptic soy broth plates, this standard medium being amended with 5 g per Liter of Tween 80 and 1 g of Soybean lecithin. Plates were incubated at 36° C. for a period 48 hours. The bacteria were then swabbed from the plates and suspended in 4 ml of Müller-Hinton broth amended with 100 mg of Tween 80 per Liter (MH-Tween) and incubated again at 36° C. for 16 hours. Following incubation the bacterial suspensions were diluted in MH-Tween to obtain a final cell density of 10⁶ colony forming units per ml. For each fragrance material four microtiter plates were used in the example, each microtiter plate having 8 rows, A-H, and 12 columns, 1-12.

To each column a diluted suspension of a different test organism was distributed, 100 μl per well. 1% stock solutions of the fragrance materials were then prepared in MH-Tween broth. The fragrance material was dispersed by ultrasonication into the aqueous medium to obtain a homogenous emulsion. 100 μl per well of this emulsion was then added to the first row of the microtiter plates already containing the target organisms. Serial dilution series were then prepared starting at row A and continuing until row G, each time removing 100 μl from a well and transferring it to the next well. In this way fragrance concentrations of 0.5%, 0.25%, 0.125%, 0.0625%, 0.03125%, 0.0156%, and 0.0078% were tested for their antibacterial efficacy. The reference compound Triclosan was tested at 16-fold lower concentrations. The plates were covered with plastic films and incubated for 24 h at 36° C. with shaking at 250 rpm. The turbidity developing in the microtiter plates was then examined after 24 h to determine microbial growth. The minimal concentration of fragrance inhibiting the growth of an organism by at least 80% was determined as the minimal inhibitory concentration (MIC).

TABLE 1
Minimal inhibitory concentration (MIC) for bacteria
derived from the human axilla of relevant perfume raw materials
described in this study. Data are expressed in % weight/volume

| | Ax 25 | Ax 26 | Ax 3 | Ax 7 | Ax 10 | Ax 11 | Ax 12 | Ax 15 | Ax 20 | Ax 19 |
|---|---|---|---|---|---|---|---|---|---|---|
| Sandela | 0.0234 | 0.0156 | 0.0176 | 0.0273 | 0.0137 | 0.0273 | 0.0137 | 0.0234 | 0.0273 | 0.0086 |
| Farnesol | 0.0156 | 0.0078 | 0.0156 | 0.0137 | 0.0078 | 0.0195 | 0.0137 | 0.0156 | 0.0137 | 0.0156 |
| Sandal- | 0.0313 | 0.0156 | 0.0195 | 0.0313 | 0.0164 | 0.0313 | 0.0313 | 0.0313 | 0.0313 | 0.0313 |
| wood oil | | | | | | | | | | |
| Sandalore | 0.1250 | 0.1250 | 0.1250 | 0.1250 | 0.1250 | 0.1250 | 0.1250 | 0.2500 | 0.1250 | n.d. |
| Radjanol | 0.1250 | 0.0625 | n.t. | 0.0625 | n.t. | n.t. | n.t. | 0.125. | n.t. | n.t. |
| Ebanol | 0.1250 | 0.1250 | 0.1250 | 0.1250 | 0.2500 | 0.2500 | 0.2500 | 0.2500 | 0.1250 | n.d. |
| Triclosan | 0.000015 | 0.0015 | 0.0029 | 0.0029 | 0.0020 | 0.0029 | 0.0017 | 0.0029 | 0.0107 | 0.0039 |

Ax 25 was identified as Staphylococcus. epidermidis by FAME analysis. Identification with the Api Coryne test kit yielded the following species assignments for the remainder of the strains: Ax 26 Corynebacterium sp.; Ax 3 Corynebacterium bovis; Ax 7 Corynebacterium group G; Ax 10 Corynebacterium jeikeium; Ax 11 Corynebacterium jeikeium; Ax 12 Corynebacterium jeikeium; Ax 15 Corynebacterium jeikeium; Ax 20 Corynebacterium striatum/amycolatum; and Ax 19 Corynebacterium jeikeium. The strains were isolated from the axilla of 8 human volunteers. Based on biochemical tests and colony morphology all strains were distinct from each other, even the several strains all belonging to Corynebacterium jeikeium.

### Example 2

Antibacterial Activity Against Brevibacterium epidermidis

A selection of perfume compounds were tested against Brevibacterium epidermidis DSMZ 9586 (German Collection of Microorganisms and Cell Cultures) with the method described in Example 1. The MIC value obtained for Sandela is comparable with the values reported for the axilla bacteria, and it is thus appropriate to use this perfume compound also in deodorant products intended to prevent the cheesy smell of feet and shoes.

MIC (% w/v) for Brevibacterium epidermidis

| | |
|---|---|
| Sandela | 0.0156 |
| Sandalore | 0.0937 |
| Sandalwood oil | 0.0156 |
| Radjanol | 0.03125 |
| Farnesol | 0.0078 |
| Triclosan | 0.003125 |

### Example 3

Antibacterial Activity Against Propionibacterium Acnes

A selection of perfume compounds were tested against Propionibacterium acnes DSMZ1897 (German Collection of Microorganisms and Cell Cultures) with the method described in Example 1, with the exception that the organism was grown under anaerobic conditions during 72 hours until evaluation of results.

MIC (% w/v) for Propionibacterium acnes

| | |
|---|---|
| Sandela | 0.03125 |
| Sandalore | 0.25 |
| Sandalwood oil | 0.0156 |
| Farnesol | 0.0078 |
| Triclosan | 0.00039 |

### Example 4

Comparison of the Antibacterial Action of Perfume Oils with High Sandela and Farnesol Content

Perfume compositions with high Sandela and/or Farnesol content were prepared as described in Table 2. These are typical perfumes to be incorporated in cosmetic products with deodorant or antiperspirant action. The MIC of these perfume compositions against a selection of the test organisms was tested with the method described in Example 1. The results are summarized in Table 3. Indeed these perfumes have a MIC between 0.025% and 0.05% which indicates strong antibacterial activity. Furthermore it becomes evident from these data, that Farnesol can be partially or fully replaced with Sandela in a perfume composition without loosing the activity significantly. Finally, adding Farnesol in addition to Sandela further enhances the activity of such compositions.

TABLE 2
Composition of deodorant perfumes (parts per weight)

| | composition | | | |
|---|---|---|---|---|
| | A | B | C | D |
| ACET CEDRENYL 70 | 60 | 60 | 60 | 43 |
| ACET LINALYLE SYNT | 30 | 30 | 30 | 21 |
| ACET P T BUTYL CYCLOHEXYLE | 55 | 55 | 55 | 39 |
| ADOXAL at 10% in DPG | 2 | 2 | 2 | 1 |
| ALD PHENYL ACETIQUE 85%/APE | 10 | 10 | 10 | 7 |
| ALLYL AMYL GLYCOLATE | 12 | 12 | 12 | 9 |
| AMBROFIX at 10% in DPG | 4 | 4 | 4 | 3 |
| BRASSYLATE ETHYLENE | 20 | 20 | 20 | 14 |
| CEDRYL METHYL ETHER | 20 | 20 | 20 | 14 |
| CITRAL SYNT 80%/ORANGE TERPENES | 1 | 1 | 1 | 1 |
| DIHYDRO MYRCENOL | 130 | 130 | 130 | 93 |
| EVERNYL | 1 | 1 | 1 | 1 |
| FARNESOL | 400 | 0 | 200 | 200 |
| GERANIOL | 20 | 20 | 20 | 14 |
| GERANIUM ESS AFRIQUE | 20 | 20 | 20 | 14 |
| ISO E SUPER | 100 | 100 | 100 | 71 |
| LILIAL | 120 | 120 | 120 | 86 |
| LINALOL SYNT | 50 | 50 | 50 | 37 |
| SALICYLATE HEXENYLE-3-CIS | 15 | 15 | 15 | 11 |
| SANDELA | 0 | 400 | 200 | 400 |
| TROPIONAL | 30 | 30 | 30 | 21 |
| Total | 1100 | 1100 | 1100 | 1100 |

TABLE 3
Antibacterial activity of deodorant perfumes

| | composition | | | |
|---|---|---|---|---|
| | A | B | C | D |
| Ax 25 | 0.0625 | 0.0625 | 0.0625 | 0.03125 |
| S. epidermidis | | | | |
| Ax 26 | 0.02343 | 0.0625 | 0.0117 | 0.0156 |
| Corynebacterium xerosis | | | | |
| Ax 7 | 0.03125 | 0.03125 | 0.03125 | 0.03125 |
| Corynebacterium | | | | |
| group G | | | | |
| Ax 15 | 0.03125 | 0.03125 | 0.03125 | 0.03125 |
| Corynebacterium | | | | |
| jeikeium | | | | |
| average | 0.037108 | 0.046875 | 0.034175 | 0.027338 |

The reported concentrations are the percent (w/v) needed for at least 80% reduction in growth during 24 hours.

### Example 5

Antibacterial Activity of Deodorant Roll-On Formula Incorporating Antibacterial Perfumes

Non-alcoholic deodorant compositions for Roll-on deodorants were prepared as described in Table 4. The Deodorants were amended with 1.4% of the perfumes according to the present invention which were described in Example 4. The antibacterial activity of the deodorants was determined according to the procedure described in Example 1. Instead of using emulsions of the perfume products in MH-Tween, 100 μl of the finished deodorant was added to the first well of the Microtiter plates containing the target organisms. Dilution series of this finished product were thus prepared in MH-Tween, and growth of the microorganisms in these product solutions were monitored.

TABLE 4
Composition of Deodorants containing
perfumes according the present invention

| | |
|---|---|
| Hydroxyethylcellulose | 0.30 |
| Water | 62.1 |
| Dipropylene Glycol | 30.00 |
| Propylene Glycol | 5.00 |
| PEG 40 hydrogenated Castor oil | 2.50 |
| Tetrasodium EDTA | 0.10 |
| Citric acid | qsp pH = 5.50 |
| Perfume | 1.4 |

Hydroxyethylcellulose was dispersed in water at 35° C. until homogeneous. PEG 40 hydrogenated Castor oil was mixed with dipropylene glycol and propylene glycol and this mixture was added. The remaining constituents were then added.

TABLE 5
Growth inhibitory effect of the Deodorant formula according to
table 4 with the perfume compositions described in Example 4.

| | no | 1.4% | 1.4% | 1.4% |
|---|---|---|---|---|
| | perfume | perfume B | perfume C | perfume A |
| Staphylococcus | 11 | 38 | 32 | 64 |
| epidermidis Ax 25 | | | | |
| Corynebacterium | 10 | 45 | 45 | 128 |
| xerosis Ax 26 | | | | |
| Corynebacterium | 27 | 54 | 54 | 128 |
| group G Ax10 | | | | |
| Corynebacterium | 27 | 54 | 32 | 64 |
| jeikeium Ax 15 | | | | |

Reported are the maximal dilutions of the product which still inhibits the growth of the axilla bacteria

### Example 6

Further Examples of Deodorants

The following sets forth examples for the use of perfume compositions according to the present invention in various products. The methods of preparing the following compositions are well known to those skilled in the art. All formulations may contain additional ingredients known to those skilled in the art, e.g., colorants, opacifiers, buffers, antioxidants, vitamins, emulsifiers, UV absorbers, silicones and the like. All products can also be buffered to the desired pH. All values are % w/w.

A) Deo-colognes:

| | |
|---|---|
| Perfume | 0.5-10.0 |
| Ethanol | to 100.0 |

B) Deo-Sticks:

| | |
|---|---|
| Antiperspirant stick: | |
| Ethylene Glycol Monostearate | 7.0 |
| Shea butter | 3.0 |
| Neobee 1053 (PVO International) | 12.0 |
| Generol 122 (Henkel) | 5.0 |
| Kesscowax B (Akzo) | 17.0 |
| Dimethicone Dow Corning 345 | 35.0 |
| Aluminum Sesquichlorhydrate | 20.0 |
| Perfume | 1.0 |
| Clear Deodorant Stick | |
| Witconol APM | 43.0 |
| Propylene Glycol | 20.0 |
| Alcohol 39C | 21.0 |
| Water | 7.0 |
| Monamid 150ADD | 5.0 |
| Millithix 925 | 2.0 |
| Ottasept Extra | 0.5 |
| Perfume | 1.5 |
| Antiperspirant Aerosol | |
| Absolute Ethanol | 15.0 |
| Zirconium Aluminum tetrachlorhydrate | 5.0 |
| Bentone 38 | 1.5 |
| Perfume | 1.25 |
| S-31 Hydrocarbon propellant | to 100.0 |
| Roll-On | |
| Dimethicone DC 354 (Dow Corning) | 69.0 |
| Bentone 38 | 10.0 |
| Rezal 36 GP (Reheis Chem. Co.) | 20.0 |
| Perfume | 1.0 |

In all these compositions, the perfume contains from 10-60% of Sandela and from 0-50% of Farnesol.

In the above, the following components were used:

| | |
|---|---|
| Neobee 1053 | glycerol tricaprate/caprylate |
| Generol 122 | soya sterol |
| Kesscowax B | cetyl alcohol and glycol polymer |
| Witconol APM | polypropylene glycol-3 myristyl ether |
| Monamid 150ADD | cocoamide diethanolamine |
| Millithix 925 | dibenzylidene sorbitol |
| Ottasept Extra | quaternium 18 hectorite |
| Bentone 38 | quaternium 18 hectorite |
| Dimethicone DC 354 | mixture of fully methylated linear |
| | siloxane polymers end-blocked with |
| | trimethylsiloxy units |
| Rezal 36 GP | aluminum |
| | zirconiumtetrachlorohydrexglycine |

### Example 7

Comparison of the Antibacterial Activity of Sandela Distillation Fractions with Varying Content of Different Sandela Isomers

Since sandela is known to be a mixture of different reaction products, commercial sandela was subjected to a distillation process. A total of 16 fractions were obtained in a distillation of 2350 g of commercial sandela over a 1.3 m column at a pressure of 0.1 Torr. The different fractions were evaluated for their antimicrobial activity (Table 6) with the method described in example 1. From these results it appears, that the late fractions 14-15 are more active than the early fraction 2-3. Even more active was the last fraction 16, which obviously contains the most active compounds. GC-MS analysis of the single fractions led to the conclusion, that fractions 14-15 contain different isomers of 3-(5,5,6-trimethylbicyclo[2.2.1]hept-2-yl)-cyclohexanol and 4-(5,5,6-trimethylbicyclo[2.2.1]hept-2-yl)-cyclohexanol while fraction 2-3 have a high content of 2-(5,5,6-trimethylbicyclo[2.2.1]hept-2-yl)-cyclohexanol. The main peaks in fraction 16 could be attributed to neither of the above mentioned isomers.

TABLE 6
Minimal inhibitory concentration (MIC) for
different fractions obtained from sandela by distillation
(data are expressed in % weight/volume)

| | | | Corynebacteria |
|---|---|---|---|
| | Boiling point | | (average for three |
| | (° C.) | S. epidermidis | organisms) |
| sandela | | 0.023 | 0.022 |
| Fraction | 109-119_{0.1 Torr} | 0.063 | 0.077 |
| 1-2 | | | |
| Fraction | 131_{0.08 Torr} | 0.022 | 0.022 |
| 14-15 | | | |
| Fraction | >131_{0.08 Torr} | 0.008 | 0.008 |
| 16 | | | |

### Example 8

Isolation and Structure Elucidation of Compounds from Most Active Distillation Fraction of Sandela

As can be seen from Example 7, the latest fraction (16) of the distillation process contains the most active products. The main peaks in the gas chromatogram within this sample were subjected to GC-MS analysis and it was found that they do not correspond to sandela isomers but that their mass spectrum would conform to compounds having an additional methoxy group (=methoxysandela): 2-methoxy-4-(5,5,6-trimethylbicyclo[2.2.1]hept-2-yl)-cyclohexanol and 2-methoxy-5-(5,5,6-trimethylbicyclo[2.2.1]hept-2-yl)-cyclohexanol. This sample was therefore again subjected to a fractional distillation and the single fractions were examined for antibacterial activity and compared with GC-MS analysis. Fractions with increased methoxysandela content had the highest activity.

Therefore the fraction having both the highest activity and the highest content of methoxysandela isomers was subjected to column chromatography with silica resin (0.063-0.2 mm) using hexane/methyl-tert-butyl ether (1:1) as eluent. Chromatography fractions which contained only methoxysandela isomers were pooled. The resulting sample still contained three peaks when evaluated with gas chromatography. These peaks were separated with preparative gas chromatography and the structure was elucidated with NMR. The spectra conformed to the following structures:

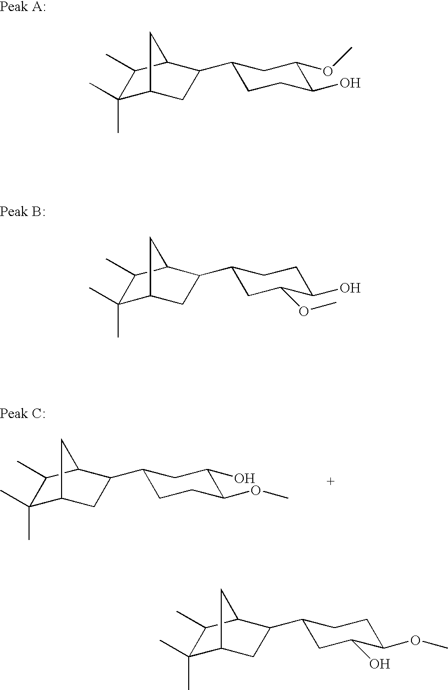

Finally, the different isomers obtained by preparative GC and some distillation and chromatography fractions from the separation procedure were compared for their minimal inhibitory concentration as described in example 1. From the results shown in Table 7 it can be seen that the most active isomers have an antimicrobial activity which is comparable to Triclosan when measured against the malodor forming bacteria colonizing the human skin.

TABLE 7
Bacteriostatic activity for distillation fractions
and pure compounds obtained from sandela
compared to benchmarks

| | | Corynebact. | Corynebact. | Corynebact. |
|---|---|---|---|---|
| | S. epidermidis | Ax 26 | Ax 7 | Ax 15 |
| sandela | 0.023 | 0.0156 | 0.0273 | 0.0234 |
| Fraction 16 | 0.0131 | 0.0086 | 0.0156 | 0.0130 |
| Mixture of | 0.0083 | 0.0048 | 0.0083 | 0.0083 |
| Peak A, B | | | | |
| and C* | | | | |
| Peak A | 0.0040 | 0.0028 | 0.0048 | 0.0048 |
| Peak B | 0.0023 | 0.0016 | 0.0028 | 0.0028 |
| Peak C | 0.0083 | 0.0048 | 0.0083 | 0.0083 |
| Farnesol | 0.0156 | 0.0078 | 0.0137 | 0.0156 |
| Triclosan | 0.000015 | 0.0025 | 0.0025 | 0.0030 |

*obtained by column chromatography

The reported concentrations are the percent (w/v) needed for at least 80% reduction in growth during 24 hours.

The invention being thus described, it will be obvious that the same may be varied in many ways. Such variations are not to be regarded as a departure from the spirit and scope of the invention and all such modifications are intended to be included within the scope of the following claims.

1. A method of providing antibacterial activity, to a patient in need thereof, by administering an antibacterially effective amount of a composition comprising one or more compound(s) of formula I 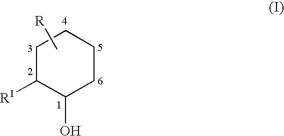
wherein formula I R is a residue of the formula II 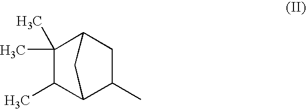
and
R is located at position 2, 3, or 6, and R1 is hydrogen; or
R is located at position 4, and R¹ is hydrogen or methoxy; or
R is located at position 5, and R¹ is methoxy; and
a pharmaceutically acceptable carrier.

2. The method of claim 1, wherein the composition contains the compound 2-methoxy-4-(5,5,6-trimethylbicyclo[2.2.1]hept-2-yl)cyclohexan-1-ol.

3. The method of claim 1, wherein the composition contains the compound 2-methoxy-5-(5,5,6-trimethylbicyclo[2.2.1]hept-2-yl)cyclohexan-1-ol).

4. The method of claim 1, wherein the composition comprises one of more compound(s) that are a mixture of at least 2-methoxy-4-(5,5,6-trimethyl-bicyclo[2.2.1]hept-2-yl)cyclohexan-1-ol and 2-methoxy-5-(5,5,6-trimethylbicyclo[2.2.1]hept-2-yl)cyclohexan-1-ol.

5. The method of claim 1, wherein the composition comprises of 0.1 to 1% by weight of the compound of formula I.

6. The method of claim 1, wherein the composition comprises of 0.3 to 0.6% by weight of the compound of formula I.

7. The method of claim 1, wherein the composition further comprises of 3,7,11-trimethyl-2,6,10-dodecatrien-1-ol.

8. The method of claim 1, wherein the composition contains a perfume, about 10 to about 80% by weight of which perfume is composed of a compound of formula I.

9. The method of claim 1, wherein the composition contains a perfume, about 10 to about 80% by weight of which perfume is composed of a compound of formula I, and wherein the compound is the only antibacterial agent in the composition.

10. The method of claim 1, wherein the composition contains a perfume, about 10 to about 80% by weight of which perfume is composed of a compound of formula I, and from about 5 to about 50% by weight of the composition is 3,7,11-trimethyl-2,6,10-dodecatrien-1-ol.

11. The method of claim 1, wherein the composition further comprises of an ingredient selected from the group consisting of water, dipropylene glycol, propylene glycol, and combinations thereof.

12. The method of claim 1, wherein the antibacterial activity results in reduction of malodor.

13. The method of claim 1, wherein the antibacterial activity results in the treatment of acne.

14-18. (canceled)
